(19) United States
(12) Patent Application Publication
(10) Pub. No.: US 2025/0152586 A1
(43) Pub. Date: May 15, 2025

(54) PHARMACEUTICAL COMPOSITION FOR RELIEVING OR TREATING PAIN BY REGULATING CEREBELLAR GLIAL CELLS AND METHOD FOR RELIEVING OR TREATING PAIN

(71) Applicants: Seoul National University R&DB Foundation, Seoul (KR); University-Industry Cooperation Group of Kyung Hee University, Gyeonggi-do (KR)

(72) Inventors: Sang Jeong KIM, Seoul (KR); Sun Kwang KIM, Seoul (KR); Seung Ha KIM, Seoul (KR)

(73) Assignees: Seoul National University R&DB Foundation, Seoul (KR); University-Industry Cooperation Group of Kyung Hee University, Gyeonggi-do (KR)

(21) Appl. No.: 18/720,471

(22) PCT Filed: Dec. 13, 2022
(86) PCT No.: PCT/KR2022/020234

§ 371 Date: Feb. 4, 2025

(30) Foreign Application Priority Data
Dec. 16, 2021 (KR) 10-2021-0180671

(51) Int. Cl. A61K 31/517; A61K 31/42; A61N 1/36; A61P 25/04; C12N 15/113

(52) U.S. Cl. CPC A61K 31/517; A61K 31/42; A61N 1/36071; A61P 25/04; C12N 15/1138; C12N 2310/531

(57) ABSTRACT

The present invention relates to a composition for relieving or treating pain, a method for relieving or treating pain by using same, and a method for screening for a pain-relieving substance. It has been confirmed that pain can be relieved by regulating the cerebellum, whose role in pain has not been known so far, by using the pharmaceutical composition and the method for relieving pain, according to the present invention. Therefore, the cerebellum can be used as a new treatment target for pain relief through the pharmaceutical composition and the method for relieving pain, according to the present invention. It has also been confirmed that, even when the cerebellum is used as a target for pain according to the present invention, there is no impact on motor function. Thus, the pharmaceutical composition and the method can be effectively used in practice in relieving or treating pain.

### TECHNICAL FIELD

The present invention relates to a composition for relieving or treating pain, a method for relieving or treating pain using the same, and a method for screening for a pain-relieving substance.

Meanwhile, the present application was supported by the following national research and development project.

### NATIONAL RESEARCH AND DEVELOPMENT PROJECT SUPPORTING PRESENT INVENTION

[Project identification No.] 1711158196

[Project No.] 2018R1A5A2025964

[Name of department] Ministry of Science and ICT

[Name of project management (professional) institution] National Research Foundation of Korea

[Research program title] Group research support

[Research project title] Memory network research center

[Contribution rate] 80/100

[Name of institution performing the project] Seoul National University

[Research period] Mar. 1, 2022 to Feb. 28, 2023

[Project identification No.] 1345252914

[Project No.] NRF-2016R1D1A1A02937329

[Name of department] Ministry of Education

[Name of project management (professional) institution] National Research Foundation of Korea

[Research program title] Personal Basic Research Support for Science and Engineering

[Research project title] The role of Bergmann glia in the processing of cerebellar pain

[Contribution rate] 10/100

[Name of institution performing the project] Kyung Hee University

[Research period] Nov. 1, 2016 to Oct. 31, 2019

[Project identification No.] 1711052808

[Project No.] NRF-2017M3C7A1025604

[Name of department] Ministry of Science and ICT

[Name of project management (professional) institution] National Research Foundation of Korea

[Research program title] Brain Science Fundamental Technology Development

[Research program title] Development of core technology for pain evaluation & control based on action mechanism/plasticity of cortical neural circuit

[Contribution rate] 10/100

[Name of institution performing the project] Kyung Hee University

[Research period] May 1, 2017 to Dec. 31, 2021

### BACKGROUND ART

Pain is a sensation induced by actual or potential tissue damage, and means both the body's preventive response and a common clinical symptom of many diseases. Pain typically evokes avoidance or protective behavior in an animal or human to remove or protect himself from further exposure to danger. Most pain resolves once the noxious stimulus is removed and the body has healed, but it may persist despite removal of the stimulus and apparent healing of the body.

Meanwhile, the cerebellum is a part of the brain that is prominently observed in the hindbrain of most vertebrates, and its main functions revealed to date are to play an important role in integrating sensory perceptions and coordinating and controlling motor muscles.

Further, the locus coeruleus (LC), which is known to be involved in arousal, attention, emotion, pain, and the like, secretes noradrenaline (NA) to all regions of the brain to regulate the noradrenaline (LC-NA system). In this case, NA becomes active depending on the NA receptor expressed in each cell, and typically, cells are activated through alpha-1 adrenergic receptors.

Korean Patent Publication No. 10-1912460 relates to a method for restoring the analgesic effect of an a2-adrenergic receptor agonist in the treatment of neuropathic pain, in which pain is relieved using an a2-adrenergic receptor agonist, a regulator of G-protein signaling (RGS) inhibitor, or an endocytosis inhibitor, but does not disclose that the cerebellum can be used as a target for pain.

### DISCLOSURE

### Technical Problem

Thus, the present inventors have made efforts to use the cerebellum as a new target for pain relief and treatment, and as a result, confirmed that pain can be remarkably relieved or treated by treating the cerebellum with an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist, thereby completing the present invention.

The present invention is directed to providing a pharmaceutical composition for relieving or treating pain, including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist.

The present invention is also directed to providing a method for relieving or treating pain, the method including administering the pharmaceutical composition to a non-human individual.

The present invention is also directed to providing a method for producing an animal model in which pain is relieved.

The present invention is also directed to providing a method for screening for a pain-relieving substance.

The present invention is also directed to providing an electronic medicine for pain relief.

The present is also directed to providing a method for relieving or treating pain, the method including administering a composition including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist to an individual in need thereof.

The present invention is also directed to providing a use of one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist for preparing a drug for relieving or treating pain.

The present invention is also directed to providing a method for relieving or treating pain by inhibiting cerebellar glial cells, the locus coeruleus, cerebellar glial cell noradrenergic receptors or cerebellar nuclei.

### Technical Solution

The terms used in the present specification are used for descriptive purposes only and should not be construed as limiting. Singular expressions include plural expressions unless the context clearly indicates otherwise. In the present specification, it should be understood that the term “include” or “have” is intended to designate the presence of features, numbers, steps, operations, constituent elements, and parts described in the specification or combinations thereof, and does not exclude in advance the possibility of the presence or addition of one or more other features, numbers, steps, operations, constituent elements, and parts, or combinations thereof. Unless defined otherwise, all terms used herein, including technical or scientific terms, have the same meaning as commonly understood by a person with ordinary skill in the art to which exemplary embodiments pertain. Terms such as those defined in commonly used dictionaries should be interpreted as having a meaning consistent with the meaning in the context of the relevant art and should not be interpreted in an ideal or overly formal sense unless explicitly defined in the present application.

One aspect of the present invention provides a pharmaceutical composition for relieving or treating pain, including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist.

In an exemplary embodiment of the present invention, the pharmaceutical composition may be a pharmaceutical composition for relieving or treating pain, which exhibits one or more activities selected from the group consisting of the following (i) to (iv):
(i) inhibition of the axon terminals of neurons in the locus coeruleus;
(ii) inhibition of adrenergic receptors to interfere with the action of noradrenaline secreted from the axon terminals of neurons in the locus coeruleus on cerebellar glial cells;
(iii) inhibition of calcium activation in cerebellar glial cells; and
(iv) inhibition of the cerebellar nuclei, a subpathway for the activation of cerebellar glial cells.

In an exemplary embodiment of the present invention, the pain may be neuropathic pain, acute pain, neurogenic pain, or a combination thereof.

In a preferred exemplary embodiment of the present invention, the noradrenergic receptor inhibitor may be an alpha−1 adrenergic blocking agent.

In an exemplary embodiment of the present invention, the alpha-1 adrenergic receptor inhibitor (alpha-1 adrenergic blocking agent) is a substance that inhibits the expression or activity of cerebellar glial cell-specific alpha-1 adrenergic receptors, and the inhibitor may be one or more selected from the group consisting of small interference RNA (siRNA), short hairpin RNA (shRNA), microRNA (microRNA), a ribozyme, a DNAzyme, a peptide nucleic acid (PNA), an antisense oligonucleotide, and an antibody.

In an exemplary embodiment of the present invention, the alpha-1 adrenergic receptor inhibitor (alpha-adrenergic blocking agent) may include shRNA consisting of SEQ ID NO: 1.

In an exemplary embodiment of the present invention, the noradrenergic receptor inhibitor may be prazosin, and the GABAA receptor agonist may be muscimol.

In a preferred exemplary embodiment of the present invention, the pharmaceutical composition may further include a physiologically acceptable carrier.

Another aspect of the present invention provides a method for relieving or treating pain, the method including administering the pharmaceutical composition according to the present invention to a non-human individual.

Still another aspect of the present invention provides a method for screening for a pain-relieving substance by treating noradrenergic receptors with a candidate substance to confirm whether or not the noradrenergic receptors are inhibited.

In an exemplary embodiment of the present invention, when noradrenergic receptors are inhibited by treatment with the candidate substance, the method may include determining the candidate substance as a pain-relieving substance.

Yet another aspect of the present invention provides a method for screening for a pain-relieving substance by treating GABAA receptors with a candidate substance to confirm whether or not the GABAA receptors are activated.

In an exemplary embodiment of the present invention, when GABAA receptors are activated by treatment with the candidate substance, the method may include determining the candidate substance as a pain-relieving substance.

Yet another aspect of the present invention provides an electronic medicine including an electrical signal generating unit that generates an electrical signal for pain relief, in which the electrical signal is an electrical signal that inhibits (i) the axon terminals of neurons in the locus coeruleus, (ii) noradrenergic receptors activated by norepinephrine secreted from the axon terminals of neurons in the locus coeruleus, (iii) cerebellar glial cell-specific alpha-1 adrenergic receptors, and (iv) cerebellar glial cells.

In an exemplary embodiment of the present invention, the signal may be one or more selected from the group consisting of an electrical stimulus, a magnetic field, ultrasound, light, and a laser.

Yet another aspect of the present invention provides a method for relieving or treating pain, the method including administering a composition including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist to an individual in need thereof.

Yet another aspect of the present invention provides a use of one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist for preparing a drug for relieving or treating pain.

In an exemplary embodiment of the present invention, the inhibiting method may include a method of injecting a chemogenetic inhibition virus, a method of administering a local antagonist, a method of using shRNA, or a method of administering a GABAA receptor agonist which inhibits the cerebellar nuclei, but is not limited thereto.

In an exemplary embodiment of the present invention, the noradrenergic receptor inhibition is by administering prazosin, the cerebellar nucleus inhibition is by administering a GABAA receptor agonist, and the GABAA receptor agonist may be muscimol, but the present invention is not limited thereto.

### Advantageous Effects

It has been confirmed that pain can be alleviated by regulating the cerebellum, whose role in pain has not been known to date, by using the pharmaceutical composition and the method for relieving pain, according to the present invention. Therefore, the cerebellum can be used as a new treatment target for pain relief through the pharmaceutical composition and the method for relieving pain, according to the present invention. It has also been confirmed that, even when the cerebellum is used as a target for pain according to the present invention, there is no impact on motor function. Thus, the pharmaceutical composition and the method can be effectively used in practice in relieving or treating pain.

### DESCRIPTION OF DRAWINGS

FIG. 1 is a schematic view of a capsaicin-induced acute pain behavior experiment.

FIG. 1A means the measurement of licking time upon injection of capsaicin, and

FIG. 1B is a recording pattern performed to confirm a pain pattern by capsaicin.

FIG. 1C shows that licking time is increased when capsaicin is injected. FIG. 1D shows a chamber for measuring basic movement after controlling cerebellar glial activity, and FIG. 1E shows a ledge test and a gait test for measuring motor coordination.

FIG. 2 shows a method for directly expressing chemogenetic hM4Di in cerebellar glial cells and inhibiting cerebellar glial cells, and shows whether pain was reduced and the results of behavioral experiments.

FIG. 3 shows a method for expressing chemogenetic hM4D (Gi) in locus coeruleus cells which activates cerebellar glial cells and locally inhibiting only the axon terminals of the locus coeruleus in the cerebellum, and shows whether pain was reduced and the results of behavioral experiments.

FIG. 4 shows a method for locally injecting prazosin, an antagonist, into the cerebellum to inhibit alpha-1 adrenergic receptors, which are mainly expressed in cerebellar glial cells, and shows whether pain was reduced and the results of behavioral experiments.

FIG. 5 shows a method for inhibiting cerebellar glial cells by reducing the expression of alpha-1 adrenergic receptors using cerebellar glial cell-specific shRNA, and shows whether or not pain was reduced.

FIG. 6 shows a method for injecting muscimol, a GABAA receptor agonist that inhibits the cerebellar nuclei, which are the targets of cerebellar output, directly into the cerebellar nuclei, and shows whether pain was reduced and the results of behavioral experiments.

### MODES OF THE INVENTION

Hereinafter, the present invention will be described in more detail.

All technical terms used in the present invention are used in the same sense as those generally understood by one skilled in the art related to the present invention, unless otherwise defined. Further, in the present specification, a preferred method or sample is described, but those similar or equivalent thereto also fall within the scope of the present invention.

As described above, there is a need for discovering a new target region for controlling pain. Thus, the present invention presents a solution to the above-described problems by providing a pharmaceutical composition for relieving or treating pain, including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor of calcium activation in cerebellar glial cells, and a GABAA receptor agonist. The pharmaceutical composition of the present invention may be effectively used in practice in relieving or treating pain using the cerebellum as a new treatment target for pain relief by confirming that pain can be alleviated by regulating the cerebellum, whose role in pain has not been known to date.

The present invention provides a pharmaceutical composition for relieving or treating pain, including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist.

The terms used in the present invention are defined as follows.

“Treatment” refers to an approach for obtaining a beneficial or desired clinical outcome. For the purpose of the present invention, the beneficial or desired clinical outcome includes, but is not limited to, alleviation of symptoms, reduction in the extent of the disease, stabilization (that is, not worsening) of the disease state, delaying or slowing the rate of disease progression, amelioration or palliation and remission (in part or in whole) of the disease state, whether detectable or undetectable. In addition, “treatment” may refer to an increased survival rate as compared to an expected survival rate when treatment is not received. “Treatment” refers to both therapeutic treatment and prophylactic or prophylactic measures. The treatments include those required for disorders already occurring as well as disorders which are prevented. “Alleviating” a disease refers to reducing the extent and/or undesirable clinical signs of the disease state and/or slowing or prolonging the time course of progression compared to the absence of treatment. Furthermore, “treatment” may refer to an increased survival rate as compared to an expected survival rate when treatment is not received. “Treatment” refers to both therapeutic treatment and prophylactic or prophylactic measures. The treatments include those required for disorders already occurring as well as disorders which are prevented.

Cerebellar glial cells refer to the glial cells or neuroglial cells found in the cerebellum.

The locus coeruleus and the areas affected by norepinephrine are collectively called the locus coeruleus-noradrenergic system (LC-NA system). The LC-NA system functions to regulate the sleep-wake cycle, attention, memory, posture and balance, cognitive control, emotions, physiological stress, and the like.

In an exemplary embodiment, the present invention provides a pharmaceutical composition for relieving or treating pain, in which the pharmaceutical composition exhibits one or more activities selected from the group consisting of the following (i) to (iv):
(i) inhibition of the axon terminals of neurons in the locus coeruleus;
(ii) inhibition of adrenergic receptors to interfere with the action of noradrenaline secreted from the axon terminals of neurons in the locus coeruleus on cerebellar glial cells;
(iii) inhibition of calcium activation in cerebellar glial cells; and
(iv) inhibition of the cerebellar nuclei, a subpathway for the activation of cerebellar glial cells.

Specifically, in the exemplary embodiments of the present invention, as shown in Examples 2 to 6 as the method of inhibiting the activity of cerebellar glial cells, (i) a method for directly expressing chemogenetic hM4Di in cerebellar glial cells and inhibiting cerebellar glial cells, (ii) a method for expressing chemogenetic hM4D (Gi) in locus coeruleus cells which activates cerebellar glial cells and locally inhibiting only the axon terminals of the locus coeruleus in the cerebellum, (iii) a method for locally injecting the antagonist prazosin (PRZ) into the cerebellum to inhibit alpha-1 adrenergic receptors, which are mainly expressed in cerebellar glial cells, (iv) a method for inhibiting cerebellar glial cells by reducing the expression of alpha-1 adrenergic receptors using cerebellar glial cell-specific shRNA, and (v) a method for injecting muscimol, a GABAA receptor agonist that inhibits the cerebellar nuclei, which are the targets of cerebellar output, directly into the cerebellar nuclei were used.

As a result, as shown in FIGS. 2 to 6, it was confirmed that when each treatment was performed, pain was significantly reduced compared to the control.

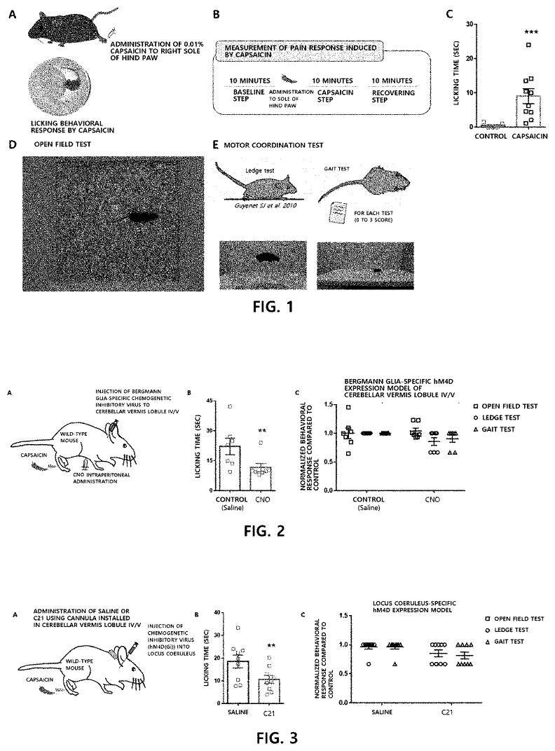

Further, since the cerebellum is the main part responsible for motor function, it is important that each treatment does not affect basic motor function. Accordingly, as shown in Example 1-3, a ledge test and a gait test were performed (see the schematic views of FIGS. 1D and 1E), and as shown in FIGS. 2 to 4 and 6, it was confirmed that basic motor function was not affected even when compared to the control.

Therefore, since it was confirmed that the pharmaceutical composition can be used to relieve and treat pain even when targeting the cerebellum and does not affect basic motor function, it was found that the cerebellum can act as a new target for pain relief and treatment.

In an exemplary embodiment, the pain may be neuropathic pain, acute pain, neurogenic pain, or a combination thereof.

The term “neuropathic pain” is used herein to refer to pain originating from abnormal processing of sensory inputs by the peripheral or central nervous system as a result of a peripheral or central nervous system disorder. In contrast to nociceptive pain, neuropathic pain is actually described as “burning,” “electric shock,” “tingling,” or “shooting.” Neuropathic pain is often described by chronic allodynia (defined as pain due to stimuli that do not normally cause a pain response, such as a light touch) and hyperalgesia (defined as increased sensitivity to normal painful stimuli), and may persist for months or years after the apparent healing of some damaged tissue.

In an exemplary embodiment of the present invention, the noradrenergic receptor inhibitor may be an alpha-1 adrenergic blocking agent.

In an exemplary embodiment, the alpha-1 adrenergic blocking agent include any substance that inhibits the expression or activity of the receptor without limitation. The alpha-1 adrenergic blocking agent inhibits the expression of cerebellar glial cell-specific alpha-1 adrenergic receptors, and the inhibitor may include one or more selected from the group consisting of small interference RNA (siRNA), short hairpin RNA (shRNA), microRNA (miRNA), a ribozyme, a DNAzyme, a peptide nucleic acid (PNA), and an antisense oligonucleotide, which are specific for alpha-1 adrenergic receptors. In addition, the alpha-1 adrenergic blocking agent is a substance that inhibits the activity of the alpha-1 adrenergic receptor, and may include one or more selected from the group consisting of an antibody, a peptide, an aptamer, and a compound, which specifically bind to the alpha-1 adrenergic receptor, but is not limited thereto. Preferably, the alpha-1 adrenergic blocking agent is an antisense oligonucleotide, an aptamer, small interference RNA (siRNA), short hairpin RNA (shRNA), microRNA (miRNA) of the genes or a compound, and most preferably short hairpin RNA (shRNA) or a compound.

According to preferred exemplary embodiments of the present invention, the shRNA sequence number may include SEQ ID NO: 1.

TABLE 1

| | | SEQ ID |
|---|---|---|
| | Sequence information (Sequence) | NO: |
| shRNA | GAGCCACACTTAGCTAACCATTTCA | 1 |

In an exemplary embodiment, the noradrenergic receptor inhibitor refers to an alpha-1 adrenergic blocking agent, and includes alfuzosin, doxazosin, terazosin, tamsulosin, prazosin, and the like, but is not limited thereto. Preferably, the noradrenergic receptor inhibitor may be prazosin.

In an exemplary embodiment, the GABAA receptor agonist includes GABA itself, muscimol, and the like as an agonist, and a barbiturate and a benzodiazepine-based agonist as a GABAA receptor positive allosteric modulator, and is not limited thereto, but may be preferably muscimol.

In the present invention, unless otherwise specified, an “agonist” is a substance that interacts with the GABAA receptor to activate the GABAA receptor and initiates the physiological or pharmacological response characteristics of the GABAA receptor, and an “antagonist” refers to a substance that competitively binds to the receptor at the same site as an agonist, but does not activate the intracellular response initiated by the active form of the receptor, thereby inhibiting the intracellular response induced by the agonist.

In an exemplary embodiment, the pharmaceutical composition may further include a physiologically acceptable carrier.

As used herein, the term “pharmaceutical composition” refers to a mixture including a compound of the present invention and a pharmaceutically acceptable excipient such as a diluent or a carrier. The pharmaceutical composition includes a cosmetic composition as well as a composition for therapeutic use. According to some exemplary embodiments, a method for administering a pharmaceutical composition including the composition of the present invention to a subject in need thereof is provided. In some exemplary embodiment, the composition of the present invention may be administered to humans.

Although the description of the pharmaceutical composition provided herein principally relates to a pharmaceutical composition for administration to humans, a person with ordinary skill in the art should understand that such a composition is generally suitable for administration to animals of all kinds. Modifications of pharmaceutical compositions for administration to a variety of animals are well understood, and a skilled veterinary pharmacologist may design and/or implement such modifications, if necessary, with no more than routine experimentation.

The pharmaceutical composition described herein may be prepared by any method known in the pharmacological field or hereinafter developed. Generally, such purification methods involve mixing an active ingredient with an excipient and/or one or more other auxiliary ingredients and then, if desired or necessary, shaping and/or packaging the product into the desired single or multiple dose units.

The pharmaceutical composition of the present invention may be prepared, packaged, and/or sold unpackaged as a single unit dose and/or a plurality of single unit doses. As used herein, “unit dose” is a discrete amount of the pharmaceutical composition including a predetermined amount of the active ingredient. The amount of active ingredient is generally equal to the dosage of active ingredient administered to a subject and/or a convenient fraction of such a dosage, such as, for example, one-half or one-third of the dosage.

The relative amounts of active ingredient, pharmaceutically acceptable excipients, and/or any additional components in the pharmaceutical composition of the invention will vary depending on the identity, size, and/or disorder of a subject to be treated, and the route by which the composition is administered. By way of example, the composition may include 0.1% to 100% (w/w) of active ingredient.

As used herein, the pharmaceutically acceptable excipient includes any and all solvents, dispersion media, diluents, or other liquid vehicles, dispersion or suspension aids, surfactants, isotonic agents, thickening agents or emulsifying agents, preservatives, solid binders, lubricants, and the like, which are suitable for the purpose of a particular dosage form. Remington's document [The Science and Practice of Pharmacy, 21st Edition, A. R. Gennaro, (Lippincott, Williams & Wilkins, Baltimore, MD, 2006] discloses various excipients that are used in the preparation of pharmaceutical compositions and known techniques for the preparation thereof. The use of any typical carrier medium is contemplated as being within the scope of the present invention, except when it is incompatible with the substance or derivatives thereof, for example, by providing any undesirable biological effect or otherwise interacting in a deleterious manner with any other components of the pharmaceutical composition. The pharmaceutically acceptable excipient is at least 95%, 96%, 97%, 98%, 99%, or 100% pure.

The pharmaceutically acceptable excipient used in the preparation of the pharmaceutical composition includes an inert diluent, a dispersant and/or a granulating agent, a surfactant and/or an emulsifying agent, a disintegrant, a binder, a preservative, a buffer, a lubricant, and/or an oil, but is not limited thereto.

A dosage form for topical and/or transdermal administration of the compound of the present invention may also include an ointment, a paste, a cream, a lotion, a gel, a powder, a solution, a spray, an inhalant and/or a patch. Generally, the active ingredient is mixed with a pharmaceutically acceptable carrier under sterile conditions and/or any necessary preservative and/or a buffer as may be required. Additionally, the present invention contemplates the use of a transdermal patch, which has the added advantage of providing controlled delivery of an active ingredient to the body. Such dosage forms may be prepared, for example, by dissolving and/or dispersing the active ingredient in a suitable medium. Alternatively or additionally, the rate may be controlled by providing a rate controlling membrane or by dispersing the active ingredient in a polymer matrix and/or gel.

Formulations for topical administration include liquid and/or semi-liquid formulations such as liniments, lotions, oil-in-water and/or water-in-oil emulsions such as creams, ointments and/or pastes, and/or solutions and/or suspensions, but are not limited thereto. Topically-administrable formulations may include, for example, about 1% to about 10% (w/w) active ingredient, although the concentration of the active ingredient may be as high as the solubility limit of the active ingredient in the solvent. Formulations for topical administration may further include one or more of the additional components described herein.

The present invention provides a method for relieving or treating pain, the method including administering the pharmaceutical composition to a non-human individual.

The present invention provides a method for relieving or treating pain, the method including administering a composition including one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist to an individual in need thereof.

In an exemplary embodiment of the present invention, the composition may exhibit one or more activities selected from the group consisting of the following (i) to (iv), but is not limited thereto:
(i) inhibition of the axon terminals of neurons in the locus coeruleus;
(ii) inhibition of adrenergic receptors to interfere with the action of noradrenaline secreted from the axon terminals of neurons in the locus coeruleus on cerebellar glial cells;
(iii) inhibition of calcium activation in cerebellar glial cells; and
(iv) inhibition of the cerebellar nuclei, a subpathway for the activation of cerebellar glial cells.

In the method for relieving or treating pain of the present invention, the type of pain, the noradrenergic receptor inhibitor, and the GABAA receptor agonist are the same as those described above, so that the description thereof will be omitted to avoid duplication.

The administration includes intravenous administration, oral or subcutaneous administration, and direct injection into the brain, and is not limited thereto, but preferably means direct injection into the cerebellum of the brain. Furthermore, a specialized drug injection mechanism for direct injection into the brain may be used, and specifically, a drug injection mechanism (Ommaya reservoir) and a drug injection catheter are included.

Further, in the treatment method of the present invention, a suitable administration amount of the drug may be applied to the administration method of the pharmaceutical composition, and accordingly, the suitable administration amount of the drug may vary depending on factors, such as the formulation method, administration method, age, body weight, gender or disease condition of a patient, diet, administration time, administration route, excretion rate and response sensitivity, and a doctor with ordinary skill may readily determine and prescribe an administration amount effective for the desired treatment or prevention. Although the amount is not limited thereto, the GABA or GABA agonist of the present invention may be administered at a concentration of 0.1 to 1.0 mmol/μl, with a single dose of 1 μl to 5 μl. When the GABA or GABA agonist of the present invention is injected at a concentration and injection amount less than the above range, the pain treatment effect is minimal, and when it is injected at a concentration and injection amount exceeding the above range, the GABAergic response may be excessively induced, resulting in side effects.

The present invention provides a method for screening for a pain-relieving substance by treating noradrenergic receptors with a candidate substance to confirm whether or not the noradrenergic receptors are inhibited.

In an exemplary embodiment of the present invention, when noradrenergic receptors are inhibited by treatment with the candidate substance, the method for screening for a pain-relieving substance may include determining the candidate substance as a pain-relieving substance.

The present invention provides a method for screening for a pain-relieving substance by treating GABAA receptors with a candidate substance to confirm whether or not the GABAA receptors are activated.

In an exemplary embodiment of the present invention, when GABAA receptors are activated by treatment with the candidate substance, the method for screening for a pain-relieving material may include determining the candidate substance as a pain-relieving substance.

The present invention provides an electronic medicine including a signal generating unit that generates a signal for pain relief, in which the electrical signal is a signal that inhibits (i) the axon terminals of neurons in the locus coeruleus, (ii) noradrenergic receptors secreted from the axon terminals of neurons in the locus coeruleus, (iii) cerebellar glial cell-specific alpha-1 adrenergic receptors, and (iv) cerebellar glial cells.

As used herein, ‘electronic medicine’ is a compound word of electronics and pharmaceuticals, and refers to an electronic device which treats diseases by regulating signals generated from the brain and nerve cells. In the present invention, ‘electronic medicine’ may be a medical device that aims to provide medicine-like therapeutic effects for various diseases including intractable diseases, by utilizing physical stimulation with minimal side effects that affect and modify the biological functions or pathological processes of the body.

Since the electronic medicine is implanted subcutaneously to selectively stimulate only a specific area that requires treatment, there is an advantage in that side effects can be minimized and in the event of a side effect, the drug effect can be immediately stopped. The site of stimulation may be a localized area of the body, specific cells, or the central or peripheral nervous system, and preferably, pain may be relieved by inhibiting the specific areas of the brain mentioned above.

The signal generating unit can be applied in any form capable of generating a signal, which may be a physical stimulus without chemical side effects. In an exemplary embodiment of the present invention, the signal is not limited to a specific form, but since biologically or chemically active ingredients are not used, there is a low risk of side effects, and the signal can be in any form capable of stimulating the specific areas of the brain mentioned above. Preferably, the signal may be one or more selected from the group consisting of an electrical stimulus, a magnetic field, ultrasound, light, and a laser.

The present invention also provides a use of one or more from the group consisting of an inhibitor of the axon terminals of neurons in the locus coeruleus, an inhibitor of noradrenergic receptors activated by norepinephrine secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist for preparing a drug for relieving or treating pain.

In the use of the present invention, the type of pain, the noradrenergic receptor inhibitor, and the GABAA receptor agonist are the same as those described above, so that the description thereof will be omitted to avoid duplication.

The present invention also provides a method for relieving or treating pain by inhibiting cerebellar glial cells, the locus coeruleus, cerebellar glial cell noradrenergic receptors or cerebellar nuclei.

In the present invention, a new target region for regulating pain was discovered, and in order to confirm this, it was confirmed that pain can be relieved by regulating the cerebellum, whose role in pain has not been known in the related art.

To confirm this, a method for inhibiting cerebellar glial cells and the locus coeruleus cells that activate cerebellar glial cells, as well as the cerebellar nuclei which are the targets of cerebellar output was specifically devised.

Therefore, in the method for relieving or treating pain, the inhibiting method may include a method of injecting a chemogenetic inhibition virus, a method of administering a local antagonist, a method of using shRNA, or a method of administering a GABAA receptor agonist which inhibits the cerebellar nuclei, but is not limited thereto.

In a specific exemplary embodiment, as the chemogenetic method, hM4D (Gi) was expressed in cerebellar glial cells (vermis lobule IV/V) and locus coeruleus cells, and the virus was activated by CNO or C21, which is an agonist capable of activating the virus to inhibit the corresponding area. In the method of administering a local antagonist, prazosin (100 μM, 0.5 μl), an alpha-1 adrenergic receptor antagonist, was used to inhibit alpha-1 adrenergic receptors, which are noradrenergic receptors mainly expressed in cerebellar glial cells, and as a method of using shRNA, cerebellar glial cell-specific shRNA was used to inhibit the alpha-1 adrenergic receptor which is a noradrenergic receptor. Furthermore, to inhibit the cerebellar nuclei, which are the targets of cerebellar output, a GABAA receptor agonist which inhibits the cerebellar nuclei was administered.

In the method of the present invention, the inhibition of the noradrenergic receptor is by administering prazosin, the inhibition of the cerebellar nuclei is by administering a GABAA receptor agonist, and the GABAA receptor agonist may be muscimol, but the present invention is not limited thereto.

In the method for relieving or treating pain of the present invention, the type of pain and the inhibition of noradrenergic receptors are the same as those described above, so that the description thereof will be omitted to avoid duplication.

### MODE FOR CARRYING OUT THE INVENTION

Hereinafter, the present invention will be described in detail through examples. However, the following examples specifically illustrate the present invention, and the contents of the present invention are not limited by the following examples.

### Example 1

### 1-1. Preparation of Animal Model

All animals were maintained on a 12-h light/dark cycle and allowed water and food ad libitum. The study was carried out in accordance with the guidelines of the Institutional Animal Care and Use Committee (IACUC) of Seoul National University. All animal experiments were carried out in accordance with IACUC-approved protocols SNU-140513-3-11 and SNU-161226-5-4.

Using non-genetically modified wild-type mice, a specific chemogenetic inhibitory virus which targets cerebellar glial cells and locus coeruleus cells was injected. To specifically remove alpha1-adrenergic receptors from cerebellar glial cells, using Glast-Cre (Tg(Slcla3-Cre/ERT) 1Nat/J), a genetically modified mouse in which Cre is expressed by tamoxifen specifically in only cerebellar glial cells, Cre was expressed by injecting tamoxifen, and then a virus that removes the alpha1-adrenergic receptor in a Cre-dependent manner was injected.

The types of reagents, animal models, types of viruses, and suppliers of reagents used in the exemplary embodiments of the present invention are as follows.

TABLE 2

| Reagent type (species) | | Source or | | Additional |
|---|---|---|---|---|
| or resource | Designation | reference | Identifiers | information |
| Strain, | Tg(Slc1a3- | Jackson | RRID: | Stock no: |
| Strain | Cre/ERT)1Nat/J | Laboratory | IMSR_JAX: | 012586 |
| background | | | 012586 | |
| Mus Musculus | | | | |
| Other | AAV9.rTH.PI.Cre.SV40 | Addgene | RRID: | Plasmid |
| | | | Addgene_107788 | #107788 |
| Other | AAV5.GFAP.hM4D(Gi).mCherry | Addgene | RRID: | Plasmid |
| | | | Addgene_50479 | #50479 |
| Other | AAV9.hSyn.DIO.hM4D(Gi).mCherry | Addgene | RRID: | Plasmid |
| | | | Addgene_44362 | #44362 |
| Other | AAV5.pSico.a1AR.shRNA.mCherry | KIST virus | | |
| | | facility | | |
| Chemical | DREADD agonist | Hellobio | CAS no: | Cat no: |
| compound, drug | 21(Compound 21) | | 2250025-92-2 | HB6124 |
| Chemical | Clozapine N-oxide (CNO) | Hellobio | CAS no: | Cat no: |
| compound, drug | dihydrochloride | | 2250025-93-3 | HB6149 |
| Chemical | Capsaicin | Sigma Aldrich | CAS no: | Cat no: |
| compound, drug | | | 404-86-4 | HB1179 |
| Chemical | Prazosin hydrochloride | Sigma Aldrich | CAS no: | Cat no: |
| compound, drug | | | 19237-84-4 | P7791 |
| Chemical | Muscimol | Sigma Aldrich | CAS no: | Cat no: |
| compound, drug | | | 2763-96-4 | M1523 |
| Chemical | Tamoxifen | Sigma Aldrich | CAS no: | Cat no: |
| compound, drug | | | 10540-29-1 | T5648 |
| Chemical | Sunflower seed oil | Santa Cruz | CAS no: | Cat no: |
| compound, drug | | Biotechnology | 8001-21-6 | sc-215936 |

1-2. Drug Injection For chemogenetic inhibition of cerebellar glial cells, CNO was injected intraperitoneally at a dose of 5 to 10 mg/kg. For chemogenetic inhibition of locus coeruleus cells, 0.5 μl of 1 mM C21 was directly injected into the cerebellar cortex through a cannula installed in cerebellar vermis lobule IV/V. Prazosin (100 μM, 0.5 μl) and muscimol (100 μM, 0.5 μl), which are alpha-1 adrenergic receptor inhibitors or GABAA agonists, were injected through cannulas targeting the cerebellar cortex and cerebellar nuclei, respectively.

### 1-3. Acute Pain Caused by Capsaicin Administration

In order to objectively measure the degree of the sense of pain in the regulation of pain, the time it took to lick the sole of the paw was measured using capsaicin which induces acute pain. This is a method for confirming patterns due to acute pain in animal models. Thereafter, it will be possible to confirm that pain can be regulated by confirming whether the time it takes to lick the sole of the paw due to capsaicin can be changed using various methods capable of regulating cerebellar glial cells.

As shown in FIG. 1A, mice were placed in a cylindrical chamber and the licking time was measured when capsaicin was injected into the sole of the paw (FIG. 1A). To confirm the pain pattern caused by capsaicin, the degree of pain was recorded for a total of 30 minutes, 10 minutes before the injection of capsaicin and 20 minutes after the injection of capsaicin (FIG. 1B).

The degree of pain was measured by measuring the time it took to lick the sole of the paw as the behavior caused by capsaicin. As a result, as shown in FIG. 1C, it was confirmed that when capsaicin was injected, the licking time significantly increased compared to a vehicle which is the control.

### 1-4. Behavioral Experiment Design

To confirm whether basic behavioral patterns were affected when the activity of cerebellar glial cells was regulated, basic movements were measured by placing the mice in a chamber measuring 40 cm in length and width (FIG. 1D).

A ledge test was first performed to measure motor coordination. The mouse was placed on the corner of the cage and its movements were measured, and when the mouse walked around the corner without losing its balance and entered the cage carefully, a score of 3 was given, and when the mouse's paws slipped a little as it walked around the corner, but otherwise appeared normal, a score of 2 was given, and when the mouse was unable to use its hind paws effectively or entered the cage with its head before its paws, a score of 1 was given, and when the mouse frequently fell off the corner and wobbled or showed no movement at all even with help, a score of 0 was given. A score closer to 3 was determined to be normal, and a score closer to 0 was determined to be abnormal motor coordination. (FIG. 1E, ledge test).

A gait test was secondly performed to measure motor coordination. An experimenter measured the mouse's walking pattern without being observed. When the mouse supported its body weight on all paws, its abdomen did not touch the floor, and both hind paws moved without any problems, a score of 3 was given, and when the mouse showed signs of trembling or dragged its paws during walking, a score of 2 was given, and the mouse showed severe trembling or limping, or a pattern in which its pelvis was lowered, or its paws were moving away from its body, a score of 1 was given, and when the mouse was unable to move forward and walked while dragging its abdomen on the floor, a score of 0 was given. A score closer to 3 was determined to be normal, and a score closer to 0 was determined to be abnormal motor coordination (FIG. 1E, gait test).

### Example 2

Method for Directly Expressing Chemogenetic hM4Di in Cerebellar Glial Cells and Inhibiting Cerebellar Glial Cells

Using non-genetically modified wild-type mice, a specific chemogenetic inhibitory virus which targets cerebellar glial cells was injected. To specifically inhibit mouse cerebellar glial cells, hM4D (Gi) was expressed in cerebellar glial cells (vermis lobule IV/V) by a chemogenetic method. DREADD uses engineered G-protein-coupled receptors (GPCRs) that are activated by ligands that selectively bind to the GPCR. For example, engineered human muscarinic type 3 GPCRs hM3D (Gq) and hM4D (Gi) do not respond to endogenous ligands but are instead activated by clozapine-N-oxide (CNO) which is pharmacologically inert. Therefore, CNO, an agonist capable of activating the corresponding virus, was injected intraperitoneally at a dose of 5 to 10 mg/kg. Behavioral experiments were performed about 1 hour after glial cells were inhibited (FIG. 2A).

As a result, in Example 1-2, when the pain induced by the above-described capsaicin was chemogenetically inhibited in a manner specific to cerebellar glial cells by intraperitoneal CNO administration, the licking time was significantly reduced compared to the control (FIG. 2B). Further, in this case, to confirm that the inhibition of cerebellar glial cells only affected the pain pattern, basic motor function was measured using the ledge test and gait test described above in Example 1-3. Basic motor-related behavioral experiments were performed and standardized based on the behavioral pattern of the control. As shown in C of FIG. 2C, due to the inhibition of cerebellar glial cells, no significance difference in basic motor performance compared to the control was confirmed. Through this, it was confirmed that by inhibiting cerebellar glial cells, pain can be reduced, and basic motor performance is not affected.

### Example 3

Expression of chemogenetic hM4D (Gi) in locus coeruleus cells which activates cerebellar glial cells and local inhibition of only the axon terminals of the locus coeruleus in the cerebellum

Using non-genetically modified wild-type mice, a specific chemogenetic inhibitory virus which targets locus coeruleus cells was injected. To specifically inhibit the locus coeruleus (LC) region, which secretes norepinephrine capable of activating mouse cerebellar glial cells, hM4D (Gi) was expressed in the LC using a chemogenetic inhibition method. Thereafter, for chemogenetic inhibition of locus coeruleus cells, 0.5 μl of 1 mM C21 was directly injected into the cerebellar cortex through a cannula installed in cerebellar vermis lobule IV/V. Behavioral experiments were performed about 10 minutes after the terminals secreting norepinephrine were inhibited (FIG. 3A). As a result, for pain induced by capsaicin, the licking time was significantly reduced compared to the control, even upon the chemogenetic inhibition of the axon terminals of locus coeruleus cells due to the local injection of capsaicin into the cerebellum through the C21 cannula (FIG. 3B). Further, in this case, to confirm that the inhibition of the axon terminals of the locus coeruleus only affected the pain pattern, basic motor function was measured using the ledge test and gait test described above in Example 1-3. Basic motor-related behavioral experiments were performed and standardized based on the behavioral pattern of the control. As shown in FIG. 3C, due to the inhibition of the axon terminals of the cerebellar locus coeruleus, no significance difference in basic motor performance compared to the control was confirmed. Through this, it was confirmed that by inhibiting the locus coeruleus, pain can be reduced, and basic motor performance is not affected.

### Example 4

Local injection of antagonist prazosin into cerebellum to inhibit alpha-1 adrenergic receptors, which are mainly expressed in cerebellar glial cells

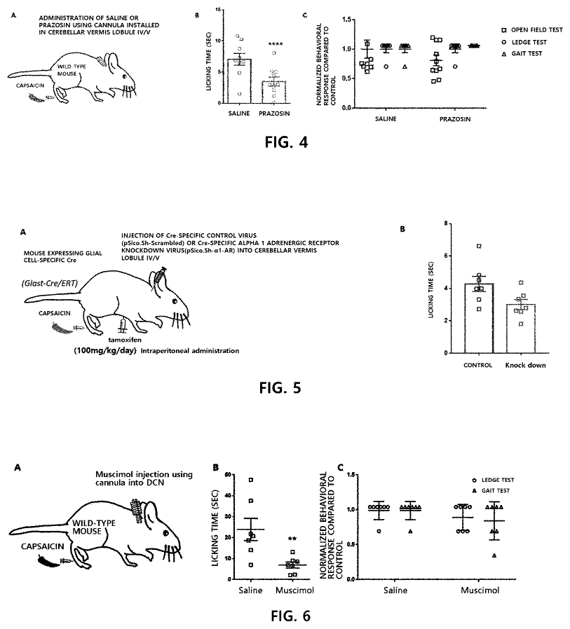

Alpha-1 adrenergic receptors in cerebellar glial cells are activated by norepinephrine. To inhibit this, prazosin (100 μM, 0.5 μl), an alpha-1 adrenergic receptor antagonist, was locally injected only into the cerebellum (vermis lobule IV/V) via a cannula. Thereafter, behavioral experiments were performed about 10 minutes after the alpha-1 adrenergic receptors were inhibited (FIG. 4A).

As a result, for pain induced by capsaicin, the licking time was significantly reduced compared to the control even upon the local injection of prazosin, an alpha-1 adrenergic receptor antagonist, into the cerebellum through a cannula (FIG. 4B). Further, in this case, to confirm that the inhibition of alpha-1 adrenergic receptors only affected the pain pattern, basic motor function was measured using the ledge test and gait test described above in Example 1-3. As shown in FIG. 4C, although cerebellar alpha-1 adrenergic receptors were inhibited, no significance difference in basic motor performance compared to the control was confirmed. Through this, it was confirmed that pain can be reduced by inhibiting alpha-1 adrenergic receptors, which are mainly expressed in cerebellar glial cells, and basic motor performance is not affected.

### Example 5

Inhibition of Cerebellar Glial Cells by Reducing the Expression of Alpha-1 Adrenergic Receptors Using Cerebellar Glial Cell-Specific shRNA

As another approach to prevent the activity of cerebellar glial cells, alpha-1 adrenergic receptors were inhibited using cerebellar glial cell-specific shRNA. To specifically remove alpha1-adrenergic receptors from cerebellar glial cells, using Glast-Cre (Tg(Slcla3-Cre/ERT) 1Nat/J), a genetically modified mouse in which Cre is expressed by tamoxifen specifically in only cerebellar glial cells, Cre was expressed by injecting tamoxifen, and then a virus that removes the alpha1-adrenergic receptor in a Cre-dependent manner was injected. Glast-cre (Tg(Slcla3-cre/ERT) 1 Nat/J) mice were purchased from Jackson Labs, USA.

Specifically, in mice expressing cerebellar glial cell-specific Cre (Glast-Cre/ERT mice), Cre is activated by estrogen derivatives, so that Cre was activated by the drug tamoxifen, and alpha-1 adrenergic receptors were knocked down in a glial cell-specific manner using Cre-dependent shRNA (534-558). The shRNA sequence used in the present invention is as follows.

TABLE 3

| | | SEQ ID |
|---|---|---|
| | Sequence information (Sequence) | NO: |
| ShRNA | GAGCCACACTTAGCTAACCATTTCA | 1 |

As a result, for pain induced by capsaicin, the licking time was significantly reduced compared to the control even upon the injection of shRNA into cerebellar glial cell-specific alpha-1 adrenergic receptors (FIG. 5B).

### Example 6

Injection of Muscimol, a GABAA Receptor Agonist that Inhibits the Cerebellar Nuclei, which are the Targets of Cerebellar Output, Directly into the Cerebellar Nuclei

Information processed in the cerebellar cortex converges to the cerebellar nuclei. To investigate the pain pattern induced by capsaicin when the cerebellar nuclei, the target of cerebellar output, were inhibited, the deep cerebellar nuclei (DCN) region was locally administered muscimol (100 μM, 0.5 μl), an inhibitory neurotransmitter GABAA receptor agonist, through a cannula. The licking time was measured 10 minutes after the injection when the effect of muscimol appeared (FIG. 6A).

As a result, for pain induced by capsaicin, the licking time was significantly reduced compared to the control due to the local injection of muscimol, a GABAA receptor agonist of DCN (FIG. 6B). Further, in this case, to confirm that the inhibitory neurotransmitter GABA receptor agonist only affected the pain pattern, basic motor function was measured using the ledge test and gait test described above in Example 1-3. As shown in FIG. 6C, due to the inhibition of the activity of cerebellar nuclei, no significance difference in basic motor performance compared to the control was confirmed. Through this, it was confirmed that by inhibiting the activity of cerebellar nuclei, pain can be reduced, and basic motor performance is not affected.

Through this, it is shown that the cerebellum can be used as a new treatment target for pain by confirming that regulating the cerebellum, whose role in pain has not been known to date, and simultaneously, there is no impact on motor function.

What is claimed is:

1. A method for relieving or treating pain, the method comprising administering a pharmaceutical composition comprising one or more from the group consisting of an inhibitor of the axon terminals of neurons in the locus coeruleus, an inhibitor of receptors of noradrenaline secreted from the axon terminals of neurons in the locus coeruleus, an inhibitor which inhibits the expression of cerebellar glial cell-specific alpha-1 adrenergic receptors, an inhibitor of calcium activation in cerebellar glial cells, and a GABAA receptor agonist; or
an electronic medicine comprising an electrical signal generating unit that generates an electrical signal for pain relief, wherein the signal is a signal that inhibits (i) the axon terminals of neurons in the locus coeruleus, (ii) receptors of noradrenaline secreted from the axon terminals of neurons in the locus coeruleus, (iii) cerebellar glial cell-specific alpha-1 adrenergic receptors, and (iv) cerebellar glial cells;
to an individual in need thereof.

2. The method of claim 1, wherein the pharmaceutical composition exhibits one or more activities selected from the group consisting of the following (i) to (iv):
(i) inhibition of the axon terminals of neurons in the locus coeruleus;
(ii) inhibition of adrenergic receptors to interfere with the action of noradrenaline secreted from the axon terminals of neurons in the locus coeruleus on cerebellar glial cells;
(iii) inhibition of calcium activation in cerebellar glial cells; and
(iv) inhibition of the cerebellar nuclei, a subpathway for the activation of cerebellar glial cells.

3. The method of claim 1, wherein the pain is neuropathic pain, acute pain, neurogenic pain, or a combination thereof.

4. The method of claim 1, wherein the inhibitor or receptors of noradrenaline is an alpha-1 adrenergic receptor inhibitor (alpha-1 adrenergic blocking agent).

5. The method of claim 4, wherein the alpha-1 adrenergic receptor inhibitor (alpha-adrenergic blocking agent) is a substance that inhibits the expression or activity of cerebellar glial cell-specific alpha-1 adrenergic receptors, and the inhibitor is one or more selected from the group consisting of small interference RNA (siRNA), short hairpin RNA (shRNA), microRNA (microRNA), a ribozyme, a DNAzyme, a peptide nucleic acid (PNA), an antisense oligonucleotide, and an antibody.

6. The method of claim 5, wherein the alpha-1 adrenergic receptor inhibitor comprises shRNA consisting of SEQ ID NO: 1.

7. The method of claim 1, wherein the inhibitor of receptors of noradrenaline is prazosin, and
the GABAA receptor agonist is muscimol.

8. The method of claim 1, wherein the pharmaceutical composition further comprises a physiologically acceptable carrier.

9. The method of claim 1, wherein the signal is one or more selected from the group consisting of an electrical stimulus, a magnetic field, ultrasound, light, and a laser.

10. (canceled)

11. A method for screening for a pain-relieving substance, comprising:
treating noradrenergic receptors with a candidate substance to confirm whether or not the noradrenergic receptors are inhibited; or
treating GABAA receptors with a candidate substance to confirm whether or not the GABAA receptors are activated.

12. The method of claim 11, comprising determining the candidate substance as a pain-relieving substance when noradrenergic receptors are inhibited or GABAA receptors are activated by treatment with the candidate substance.

13-30. (canceled)

31. A method for relieving or treating pain, the method comprising inhibiting cerebellar glial cells, the locus coeruleus, cerebellar glial cell noradrenergic receptors or cerebellar nuclei.

32. The method of 31, wherein the inhibiting comprises a method of injecting a chemogenetic inhibition virus, a method of administering a local antagonist, a method of using shRNA, or a method of administering a GABAA receptor agonist which inhibits the cerebellar nuclei.

33. The method of 31, wherein the pain is neuropathic pain, acute pain, neurogenic pain, or a combination thereof.

34. The method of 31, wherein the inhibiting the noradrenergic receptor is by administering an alpha-1 adrenergic receptor inhibitor (alpha-1 adrenergic blocking agent).

35. The method of claim 34, wherein the alpha-1 adrenergic receptor inhibitor (alpha-adrenergic blocking agent) is a substance that inhibits the expression or activity of cerebellar glial cell-specific alpha-1 adrenergic receptors, and
the inhibitor is one or more selected from the group consisting of small interference RNA (siRNA), short hairpin RNA (shRNA), microRNA (microRNA), a ribozyme, a DNAzyme, a peptide nucleic acid (PNA), an antisense oligonucleotide, and an antibody.

36. The method of claim 35, wherein the alpha-1 adrenergic receptor inhibitor comprises shRNA consisting of SEQ ID NO: 1.

37. The method of claim 31, wherein the inhibiting the noradrenergic receptor is by administering prazosin,
the inhibiting the cerebellar nuclei is by administering a GABAA receptor agonist, and
the GABAA receptor agonist is muscimol.


SEQUENCE LISTING (1 sequence)

SEQ ID NO: 1 - RNA, 25 nt, synthetic construct
  source 1..25: mol_type = other RNA
gagccacacttagctaaccatttca